(19) United States
(12) Patent Application Publication
(10) Pub. No.: US 2004/0151677 A1
(43) Pub. Date: Aug. 5, 2004

(54) 1,3,5-Triazine sunscreens solubilized in mixed phthalimide eutectics

(75) Inventor: Didier Candau, Bievres (FR)

Correspondence Address: BURNS DOANE SWECKER & MATHIS L L P, POST OFFICE BOX 1404, ALEXANDRIA, VA, 22313-1404, US

(73) Assignee: SOCIETE L'OREAL, S.A., PARIS (FR)

(21) Appl. No.: 10/735,633

(22) Filed: Dec. 16, 2003

Related U.S. Application Data
(60) Provisional application No. 60/451,241, filed on Mar. 4, 2003.

(30) Foreign Application Priority Data
Dec. 16, 2002 (FR) 02/15910

(51) Int. Cl. A61K 7/42

(52) U.S. Cl. 424/59

(57) ABSTRACT

Topically applicable cosmetic/dermatological compositions suited for UV-photoprotecting the skin, hair, lips and/or integuments against the damaging effects of UV radiation contain (a) an effective UV-photoprotecting amount of at least one 1,3,5-triazine sunscreen and (ii) an amount of at least one mixed phthalimide eutectic sufficient to solubilize the totality of the at least one 1,3,5-triazine sunscreen.

### CROSS-REFERENCE TO PRIORITY/PROVISIONAL APPLICATIONS

This application claims priority under 35 U.S.C. § 119 of FR-02/15910, filed Dec. 16, 2001, and of provisional application Serial No. 60/451,241, filed Mar. 4, 2003, both hereby expressly incorporated by reference. This application is also a continuation of said '241 provisional.

### BACKGROUND OF THE INVENTION

1. Technical Field of the Invention

The present invention relates to a photoprotective composition comprising at least one 1,3,5-triazine derivative dissolved in at least one eutectic mixture of n-butylphthalimide and of isopropylphthalimide, and also to its various cosmetic uses, especially for protecting the skin and/or the lips and/or the integuments against ultraviolet radiation, in particular solar radiation.

2. Description of Background/Related/Prior Art

It is known that light radiation with wavelengths of between 280 nm and 400 nm permits tanning of the human epidermis, and that light rays with wavelengths of between 280 nm and 320 nm, known as UV-B rays, cause skin burns and erythema which may be harmful to the development of a natural tan; this UV-B radiation should thus be screened out.

It is also known that UV-A rays, with wavelengths of between 320 nm and 400 nm, which cause tanning of the skin, are liable to induce an adverse change in the latter, especially in the case of sensitive skin or of skin which is continually exposed to solar radiation. UV-A rays cause in particular a loss of elasticity of the skin and the appearance of wrinkles, leading to premature skin aging. They promote triggering of the erythemal reaction or amplify this reaction in certain individuals and may even be the cause of phototoxic or photoallergic reactions. It is thus desirable also to screen out UV-A radiation.

Many cosmetic compositions intended for the photoprotection (UV-A and/or UV-B) of the skin have been proposed to date.

These antisun compositions are quite often in the form of an emulsion of oil-in-water type (that is to say a cosmetically acceptable support consisting of a continuous aqueous dispersing phase and a discontinuous oily dispersed phase) which contains, in various concentrations, one or more conventional lipophilic and/or hydrophilic organic screening agents which are capable of selectively absorbing harmful UV radiation, these screening agents (and their amounts) being selected as a function of the desired sun protection factor, the sun protection factor (SPF) being expressed mathematically by the ratio of the UV radiation time necessary to reach the erythema-forming threshold with the UV screening agent to the UV radiation time necessary to reach the erythema-forming threshold without UV screening agent.

1,3,5-Triazine derivatives are particularly desired in antisun cosmetics due to the fact that they are highly active in the UV-B range, and even in the UV-A range for some of these compounds depending on the nature of the substituents involved. Furthermore, they are photostable, i.e., they show little or no chemical degradation under the action of UV radiation. They are especially described in U.S. Pat. No. 4,367,390, EP-863,145, EP-517,104, EP-570,838, EP-796,851, EP-775,698, EP-878,469 and EP-933,376, and the following are known in particular:

2,4,6-tris[p-(2′-ethylhexyl-1′-oxycarbonyl)anilino]-1,3,5-triazine or “Ethylhexyl Triazone” (INCI name), sold under the trademark “Uvinul T 150” by BASF,

2-[(p-(tert-butylamido)anilino]-4,6-bis-[(p-(2′-ethylhexyl-1′-oxycarbonyl) anilino]-1,3,5-triazine or “Diethylhexyl Butamido Triazone” (INCI name), sold under the trademark “Uvasorb HEB” by Sigma 3V,

2,4-bis{[4-2-ethylhexyloxy)]-2-hydroxy]phenyl}-6-(4-methoxyphenyl)-1,3,5-triazine or “Anisotriazine” (INCI name), sold under the trademark “Tinosorb S” by Ciba Specialty Chemicals.

It has been proposed in the prior art to use 1,3,5-triazine derivatives in oils such as esters and more particularly C₁₂-C₁₅ alkyl benzoates (“Finsolv TN” from Finetex), or triglycerides and especially C₈-C₁₂ fatty acid triglycerides (“Miglyol 812” from Hüls), or alternatively oxyethylenated or oxypropylenated fatty monoalcohols or polyols (“Cetiol HE” from Henkel or “Witconol AM” from Witco). The use of these oils presents two drawbacks:

either the appearance over time of crystallization in the formulations, which is detrimental to the cosmetic qualities, the stability and the efficacy of antisun products;

or the limitation of the concentration of screening agents in the formulations, which does not make it possible to obtain products that are sufficiently effective.

The technical problem underlying the present invention is thus that of improving the photoprotective efficacy, the cosmetic properties and the stability of compositions containing such 1,3,5-triazine derivatives.

### SUMMARY OF THE INVENTION

It has now surprisingly and unexpectedly been determined that the technical problem mentioned above can be solved by adding a eutectic mixture of n-butylphthalimide and of isopropylphthalimide to a composition containing at least one triazine derivative, in an amount that is sufficient to make it possible by itself to dissolve all of the triazine derivative.

This eutectic mixture of n-butylphthalimide and of isopropylphthalimide makes it possible to obtain stable antisun compositions containing 1,3,5-triazine derivatives, which have a sun protection factor higher than those of the prior art compositions containing 1,3,5-triazine derivatives. These compositions also have improved cosmetic qualities. They especially allow good moisturization of the skin, i.e., no dryness of the skin and no greasy feel are observed.

The present invention thus features a photoprotective composition containing at least one 1,3,5-triazine derivative and at least one eutectic mixture of n-butylphthalimide and of isopropylphthalimide in a sufficient amount making it possible by itself to dissolve all of the triazine derivative.

This invention also features the use of such a composition for the manufacture of cosmetic or dermatological compositions intended in particular for protecting keratin materials against solar radiation.

The present invention also features the use of at least one n-butylphthalimide/isopropylphthalimide eutectic mixture in a photoprotective cosmetic or dermatological composition containing at least one 1,3,5-triazine derivative, in order to improve the sun protection factor and/or the cosmetic qualities and/or the stability of this composition.

Other aspects of the invention will become apparent on reading the description and the examples that follow.

### DETAILED DESCRIPTION OF BEST MODE AND SPECIFIC/PREFERRED EMBODIMENTS OF THE INVENTION

By the expression “photoprotective composition” is intended any composition containing at least one organic compound and/or at least one mineral compound capable of screening out UV-A and/or UV-B radiation via a phenomenon of absorption, reflection or scattering.

The eutectic mixture in accordance with the invention preferably comprises:

(a) 60% to 75% by weight of n-butylphthalimide of structure (1) below:

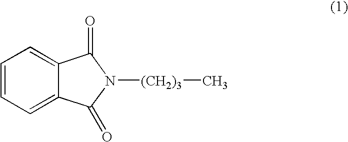

(b) 25% to 40% by weight of isopropylphthalimide in accordance with the invention, corresponding to the following structure:

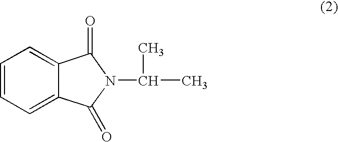

The n-butylphthalimide/isopropylphthalimide eutectic mixtures in accordance with the invention are known and are described and synthesized in U.S. Pat. No. 6,306,373 (which forms an integral part of the content of the description).

Among the eutectic mixtures that may be used, mention may be made of the following mixtures:

n-butylphthalimide/isopropylphthalimide (60/40% by weight)

n-butylphthalimide/isopropylphthalimide (62/38% by weight)

n-butylphthalimide/isopropylphthalimide (65/35% by weight)

n-butylphthalimide/isopropylphthalimide (70/30% by weight)

n-butylphthalimide/isopropylphthalimide (75/25% by weight).

The n-butylphthalimide/isopropylphthalimide eutectic mixture sold under the name Pelemol BIP by Phoenix Chemicals will be used, for example.

The eutectic mixture in accordance with the invention will preferably be used at concentrations ranging from 0.1% to 50% by weight and more preferably from 1% to 30% by weight relative to the total weight of the composition.

The 1,3,5-triazine derivative corresponds to formula (I) below:

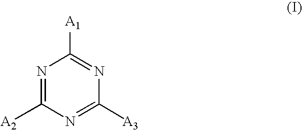

in which the radicals A₁, A₂ and A₃, which may be identical or different, are chosen from the groups of formulae (II) to (IX) below:

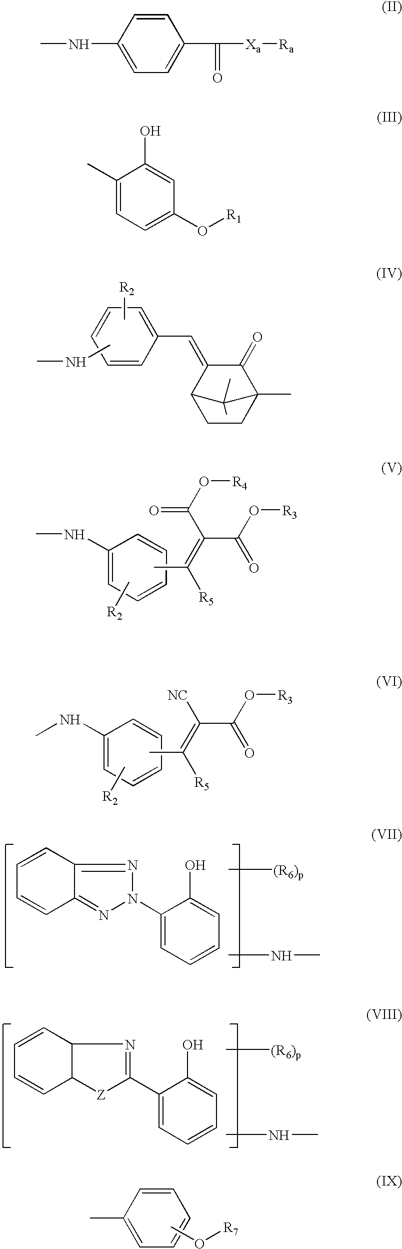

in which:

Xₐ (each of the groups Xₐ may be identical or different) represents oxygen or —NH—;

Rₐ (each of the groups Rₐ may be identical or different) is chosen from hydrogen; an alkali metal; an ammonium radical optionally substituted with one or more linear or branched C₁-C₁₈ alkyl or linear or branched C₁-C₁₈ hydroxyalkyl radicals; a linear or branched C₁-C₁₈ and preferably C₆-C₁₂ alkyl radical; a C₅-C₁₂ cycloalkyl radical optionally substituted with one or more C₁-C₄ alkyl radicals; a polyoxyethylenated radical comprising from 1 to 6 ethylene oxide units, the terminal OH group of which is methylated; a radical of formula (X), (XI) or (XII) below:

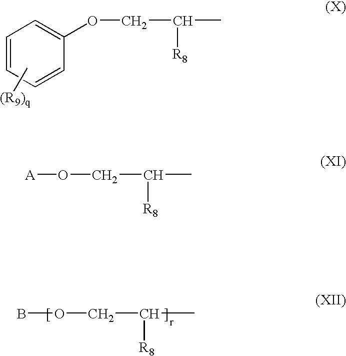

in which:

R₈ is hydrogen or a methyl radical;

R₉ is a C₁-C₉ alkyl radical;

q is an integer equal to 0; 1; 2; 3;

r is an integer equal to 1; 2; 3; 4; 5; 6; 7; 8; 9; 10;

A is a C₄-C₈ alkyl radical or a C₅-C₈ cycloalkyl radical;

B is chosen from: a linear or branched C₁-C₈ alkyl radical; a C₅-C₈ cycloalkyl radical; an aryl radical optionally substituted with one or more C₁-C₄ alkyl radicals.

R₁ denotes a C₃-C₁₈ alkyl radical; a C₂-C₁₈ alkenyl radical; a residue of formula —CH₂—CH(OH)—CH₂—OT₁ in which T₁ is a hydrogen atom or a C₁-C₈ alkyl radical; a residue of formula (XIII) below:

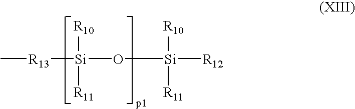

in which:

R₁₃ denotes a covalent bond; a linear or branched C₁-C₄ alkyl radical or a radical of formula —Cₘ₁H₂ₘ₁—O— in which m, is an integer equal to 1; 2; 3; 4;

p₁ is an integer equal to 0; 1; 2; 3; 4; 5;

the radicals R₁₀, R₁₁ and R₁₂, which may be identical or different, denote a C₁-C₁₈ alkyl radical; a C₁-C₁₈ alkoxy radical or a radical of formula:

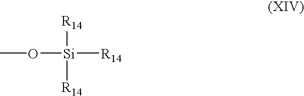

in which R₁₄ is a C₁-C₁₀ alkyl radical,

R₂ denotes a hydrogen atom, a linear or branched C₁-C₄ alkyl radical or a C₁-C₄ alkoxy radical;

R₃ and R₄, which may be identical or different, denote a linear or branched C₁-C₂₀ alkyl radical;

R₅ represents a hydrogen atom or a phenyl radical optionally substituted with a halogen or with a C₁-C₄ alkyl radical or with a C₁-C₄ alkoxy radical;

R₆ is a linear or branched C₁-C₈ alkyl radical or a C₁-C₃ alkoxy radical, it being understood that, in the latter case, two adjacent radicals R₆ on the same aromatic nucleus can together form an alkylidenedioxy group in which the alkylidene group contains 1 or 2 carbon atoms, OH, NHCOCH₃ or NH₂,

R₇ denotes a hydrogen atom, a C₁-C₁₀ alkyl radical, a radical of formula: —(CH₂CHR₅—O)ₙ₁R₈ in which n1 is a number from 1 to 16, or a radical of structure —CH₂—CH—(OH)—CH₂OT, with R₈ and T₁ having the same meaning as indicated above,

Z represents oxygen, sulfur, —NH— or —NR₃— with R₃ representing a linear or branched C₁-C₂₀ alkyl radical;

p is 0, 1, 2 or 3,

A₁ can also be a halogen, a radical —N(R₃)₂, the two radicals R₃ together possibly forming a ring of 4 or 5 carbon atoms, or a group —OR₃, R₃ having the same definition as above.

A first family of 1,3,5-triazine derivatives that is more particularly preferred, and that is described especially in EP-A-0,517,104, is that of the 1,3,5-triazines corresponding to formula (I) in which A₁, A₂ and A₃ are of formula (II) and have all of the following characteristics:

one of the groups Xₐ—Rₐ represents a radical-NH—Rₐ with Rₐ chosen from: a C₅-C₁₂ cycloalkyl radical optionally substituted with one or more C₁-C₄ alkyl radicals; a radical of formula (X), (XI) or (XII) above in which:

B is a C₁-C₄ alkyl radical;

R₉ is a methyl radical;

the other two groups Xₐ—Rₐ represent a radical —O—Rₐ with Rₐ, which may be identical or different, chosen from: hydrogen; an alkali metal; an ammonium radical optionally substituted with one or more alkyl or hydroxyalkyl radicals; a linear or branched C₁-C₁₈ alkyl radical; a C₅-C₁₂ cycloalkyl radical optionally substituted with one or more C₁-C₄ alkyl radicals; a radical of formula (X), (XI) or (XII) above in which:

B is a C₁-C₄ alkyl radical;

R₉ is a methyl radical.

A second family of 1,3,5-triazine derivatives that is more particularly preferred, and that is described especially in EP-A-0,570,838, is that of the 1,3,5-triazines corresponding to formula (I) in which A₁, A₂ and A₃ are of formula (II) and have all of the following characteristics:

one or two groups Xₐ—Rₐ represent a radical —NH—Rₐ, with Rₐ chosen from: a linear or branched C₁-C₁₈ alkyl radical; a C₅-C₁₂ cycloalkyl radical optionally substituted with one or more C₁-C₄ alkyl radicals; a radical of formula (X), (XI) or (XII) above in which B is a C₁-C₄ alkyl radical R₉ is a methyl radical;

the other or the other two group(s) Xₐ—Rₐ being a radical —ORₐ with Rₐ, which may be identical or different, chosen from: hydrogen; an alkali metal; an ammonium radical optionally substituted with one or more alkyl or hydroxyalkyl radicals; a linear or branched C₁-C₁₈ alkyl radical; a C₅-C₁₂ cycloalkyl radical optionally substituted with one or more C₁-C₄ alkyl radicals; a radical of formula (X), (XI) or (XII) above in which B is a C₁-C₄ alkyl radical and R₉ is a methyl radical.

A 1,3,5-triazine of this second family that is particularly preferred is 2-[(p-(tert-butylamido)-anilino]-4,6-bis[(p-(2′-ethylhexyl-1′-oxycarbonyl)-anilino]-1,3,5-triazine or “Diethylhexyl Butamido Triazone” sold under the trademark “Uvasorb HEB” by Sigma 3V and corresponding to the following formula:

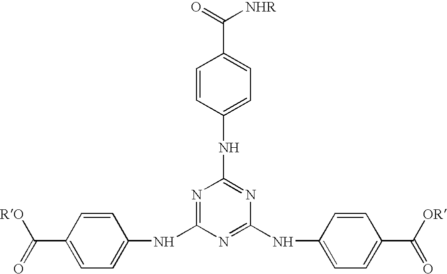

in which R′ denotes a 2-ethylhexyl radical and R denotes a tert-butyl radical.

A third preferred family of compounds that may be used in the context of the present invention, and which is described especially in U.S. Pat. No. 4,724,137, is that of the 1,3,5-triazines corresponding to formula (I) in which A₁, A₂ and A₃ are of formula (II) and have all of the following characteristics:

Xₐ are identical and represent oxygen;

Rₐ, which may be identical or different, represent a C₆-C₁₂ alkyl radical or a polyoxyethylenated radical comprising from 1 to 6 ethylene oxide units, the terminal OH group of which is methylated.

A 1,3,5-triazine of this third family that is particularly preferred is 2,4,6-tris[p(2′-ethylhexyl-1′-oxycarbonyl)anilino]-1,3,5-triazine or “Ethylhexyl Triazone” sold especially under the trademark “Uvinul T 150” by BASF and corresponds to the following formula:

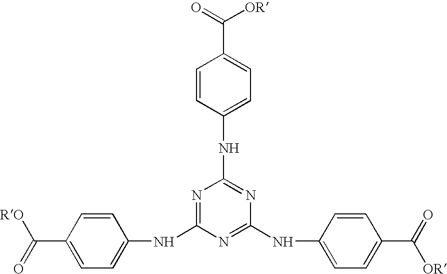

in which R′ denotes a 2-ethylhexyl radical.

A fourth preferred family of compounds that may be used in the context of the present invention, and which is described especially in EP-A-0,775,698, is that of the 1,3,5-triazines corresponding to formula (I) in which A₁ and A₂ are of formula (III) and A₃ is of formula (IX) and have all of the following characteristics: R₁, which may be identical or different, denote a C₃-C₁₈ alkyl radical; a C₂-C₁₈ alkenyl radical or a residue of formula —CH₂—CH(OH)—CH₂—OT₁ in which T₁ is a hydrogen atom or a C₁-C₈ alkyl radical; R₇ denotes a hydrogen atom or a C₁-C₁₀ alkyl radical.

A 1,3,5-triazine of this fourth family that is particularly preferred is 2,4-bis{[4-2-ethylhexyloxy)]-2-hydroxy]phenyl}-6-(4-methoxyphenyl)-1,3,5-triazine or “Anisotriazine” sold under the trademark “Tinosorb S” by Ciba Specialty Chemicals, and corresponds to the following formula:

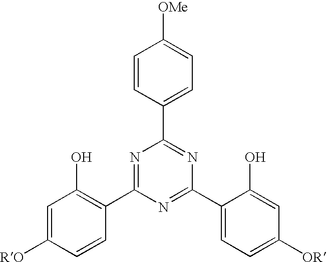

in which R′ denotes a 2-ethylhexyl radical.

A fifth preferred family of compounds that may be used in the context of the present invention, and which is described especially in EP-507,691, EP-507,692, EP-790,243 and EP-944,624, and the technical content of which is incorporated in its entirety in the present description, is that of the 1,3,5-triazines corresponding to formula (I) in which A₁, A₂ and A₃ are of formulae (VII) to (XI) mentioned above.

As examples of these compounds that may be used, mention may be made of:

2,4,6-tris(diisobutyl 4′-aminobenzalmalonate)-s-triazine,

2,4,6-tris(bis(2-ethylhexyl) 4′-aminobenzal-malonate)-s-triazine,

2,4,6-tris(bis(2-ethylhexyl) 4′-aminobenzal-malonate)-6-chloro-s-triazine,

2,4,6-tris(bis(2-ethylhexyl) 4′-aminobenzal-malonate)-6-(2-ethylhexyl 4′-aminobenzoate)-s-triazine,

2,4,6-tris(diisobutyl 4′-aminobenzalmalonate)-6-butoxy-s-triazine,

2,4,6-tris(diisobutyl 4′-aminobenzalmalonate)-6-(2-ethylhexylamino)-s-triazine,

2,4-bis(4′-aminobenzylidenecamphor)-6-(2-ethyl-hexylamino)-s-triazine,

2,4-bis(4′-aminobenzylidenecamphor)-6-(diisobutyl 4′-aminobenzalmalonate)-s-triazine,

2,4,6-tris(diethyl 4′-aminobenzalmalonate)-s-triazine,

2,4,6-tris(diisopropyl 4′-aminobenzalmalonate)-s-triazine,

2,4,6-tris(dimethyl 4′-aminobenzalmalonate)-s-triazine,

2,4,6-tris(ethyl a-cyano-4-aminocinnamate)-s-triazine,

2,4,6-tris[(3′-benzotriazol-2-yl-2′-hydroxy-5′-methyl)phenylamino]-s-triazine,

2,4,6-tris[(3′-benzotriazol-2-yl-2′-hydroxy-5′-tert-octyl)phenylamino]-s-triazine.

A 1,3,5-triazine of this fifth family that is particularly preferred is 2,4,6-tris(diisobutyl 4′-aminobenzalmalonate)-s-triazine, which corresponds to the following formula:

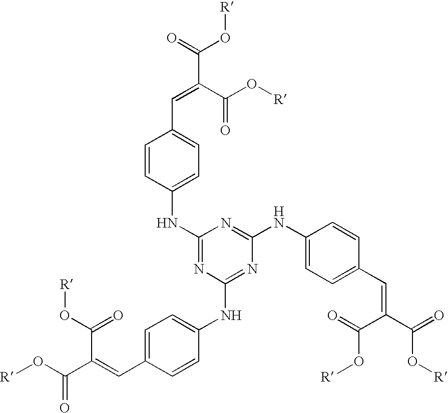

The compositions according to the present invention preferably comprise, in a physiologically acceptable medium, from 0.05% to 15% and preferably from 0.1% to 10% of 1,3,5-triazine derivatives by weight relative to the total weight of the said composition.

The said composition according to the present invention is preferably a cosmetic composition containing, besides the 1,3,5-triazine derivative as organic screening agent, at least one other additional organic screening agent and/or at least one other additional mineral screening agent, which is water-soluble, liposoluble or insoluble in the cosmetic solvents commonly used.

The compositions in accordance with the invention may also comprise other additional UVA-active and/or UVB-active organic or mineral UV-screening agents that are water-soluble or liposoluble or insoluble in the cosmetic solvents commonly used.

The additional organic screening agents are chosen especially from anthranilates; cinnamic derivatives; dibenzoylmethane derivatives; salicylic derivatives; camphor derivatives; benzophenone derivatives; β,β-diphenyl-acrylate derivatives; benzotriazole derivatives; benzalmalonate derivatives; benzimidazole derivatives; imidazolines; bis-benzazolyl derivatives as described in EP-669,323 and U.S. Pat. No. 2,463,264; p-aminobenzoic acid (PABA) derivatives; methylenebis(hydroxyphenylbenzotriazole) derivatives as described in U.S. Pat. Nos. 5,237,071, 5,166,355, GB-2,303,549, DE-197,26,184 and EP-893,119; benzoxazole derivatives as described in patent applications EP-0-832,642, EP-1-027,883, EP-1-300,137 and DE-1-0-162,844; screening polymers and screening silicones such as those described especially in WO 93/04665; dimers derived from α-alkyl-styrene, such as those described in DE-198,55,649; 4,4-diarylbutadienes such as those described in EP-0-967,200, DE-197,46,654, DE-197,55,649, EP-A-1-008,586, EP-1-133,980 and EP-133,981, and mixtures thereof.

As examples of additional organic screening agents, mention may be made of those denoted hereinbelow under their INCI name:

Para-Aminobenzoic Acid Derivatives:

PABA,

Ethyl PABA,

Ethyl dihydroxypropyl PABA,

Ethylhexyl dimethyl PABA sold in particular under the name “Escalol 507” by ISP,

Glyceryl PABA,

PEG-25 PABA sold under the name “Uvinul P25” by BASF.

Salicylic Derivatives:

Homosalate sold under the name “Eusolex HMS” by Rona/EM Industries,

Ethylhexyl salicylate sold under the name “Neo Heliopan OS” by Haarmann and Reimer,

Dipropylene glycol salicylate sold under the name “Dipsal” by Scher,

TEA salicylate sold under the name “Neo Heliopan TS” by Haarmann and Reimer.

Dibenzoylmethane Derivatives:

Butyl methoxydibenzoylmethane sold in particular under the trademark “Parsol 1789” by Hoffmann LaRoche,

Isopropyldibenzoylmethane.

Cinnamic Derivatives:

Ethylhexyl methoxycinnamate sold in particular under the trademark “Parsol MCX” by Hoffmann LaRoche,

Isopropyl methoxycinnamate,

Isoamyl methoxycinnamate sold under the trademark “Neo Heliopan E 1000” by Haarmann and Reimer,

Cinoxate,

DEA methoxycinnamate,

Diisopropyl methylcinnamate,

Glyceryl ethylhexanoate dimethoxycinnamate.

β,β-Diphenylacrylate Derivatives:

Octocrylene sold in particular under the trademark “Uvinul N539” by BASF,

Etocrylene sold in particular under the trademark “Uvinul N35” by BASF.

Benzophenone Derivatives:

Benzophenone-1 sold under the trademark “Uvinul 400” by BASF,

Benzophenone-2 sold under the trademark “Uvinul D50” by BASF,

Benzophenone-3 or Oxybenzone sold under the trademark “Uvinul M40” by BASF,

Benzophenone-4 sold under the trademark “Uvinul MS40” by BASF,

Benzophenone-5,

Benzophenone-6 sold under the trademark “Helisorb 11” by Norquay,

Benzophenone-8 sold under the trademark “Spectra-Sorb UV-24” by American Cyanamid,

Benzophenone-9 sold under the trademark “Uvinul DS-49” by BASF,

Benzophenone-12

n-hexyl 2-(4-diethylamino-2-hydroxybenzoyl)benzoate.

Benzylidenecamphor Derivatives:

3-Benzylidenecamphor manufactured under the name “Mexoryl SD” by Chimex,

4-Methylbenzylidenecamphor sold under the name “Eusolex 6300” by Merck,

Benzylidenecamphorsulfonic acid manufactured under the name “Mexoryl SL” by Chimex,

Camphor benzalkonium methosulfate manufactured under the name “Mexoryl SO” by Chimex,

Terephthalylidenedicamphorsulfonic acid manufactured under the name “Mexoryl SX” by Chimex,

Polyacrylamidomethylbenzylidenecamphor manufactured under the name “Mexoryl SW” by Chimex.

Phenylbenzimidazole Derivatives:

Phenylbenzimidazolesulfonic acid sold in particular under the trademark “Eusolex 232” by Merck,

Disodium phenyl dibenzimidazole tetrasulfonate sold under the trademark “Neo Heliopan AP” by Haarmann and Reimer.

Phenylbenzotriazole Derivatives:

Drometrizole trisiloxane sold under the name “Silatrizole” by Rhodia Chimie, Methylenebis(benzotriazolyl)tetramethylbutylphenol sold in solid form under the trademark “MIXXIM BB/100” by Fairmount Chemical, or in micronized form as an aqueous dispersion under the trademark “Tinosorb M” by Ciba Specialty Chemicals.

Anthranilic Derivatives:

Menthyl anthranilate sold under the trademark “Neo Heliopan MA” by Haarmann and Reimer.

Imidazoline Derivatives:

Ethylhexyldimethoxybenzylidenedioxoimidazoline propionate.

Benzalmalonate Derivatives:

Polyorganosiloxane containing benzalmalonate functions, as the product Polysilicone-15, sold under the trademark “Parsol SLX” by Hoffmann LaRoche

4,4-Diarylbutadiene Derivatives:

1,1-Dicarboxy(2,2′-dimethylpropyl)-4,4-diphenyl-butadiene,

Benzoxazole Derivatives

2,4-bis-[5-1 (dimethylpropyl)benzoxazol-2-yl-(4-phenyl)-imino]-6-(2-ethylhexyl)-imino-1,3,5-triazine sold under the trademark “Uvasorb K2A” by Sigma 3V, and mixtures thereof.

The preferred additional organic UV-screening agents are chosen from:

Ethylhexyl salicylate,

Ethylhexyl methoxycinnamate,

Octocrylene,

Phenylbenzimidazolesulfonic acid,

Benzophenone-3,

Benzophenone-4,

Benzophenone-5,

n-Hexyl 2-(4-diethylamino-2-hydroxybenzoyl)-benzoate,

4-Methylbenzylidenecamphor,

Terephthalylidenedicamphorsulfonic acid,

Disodium phenyldibenzimidazoletetrasulfonate,

Methylenebis(benzotriazolyl)tetramethylbutyl-phenol,

Drometrizole trisiloxane,

Polysilicone-15-

1,1-Dicarboxy(2,2′-dimethylpropyl)-4,4-diphenyl-butadiene,

2,4-bis-[5-1 (dimethylpropyl)benzoxazol-2-yl-(4-phenyl)-imino]-6-(2-ethylhexyl)-imino-1,3,5-triazine and mixtures thereof.

The additional mineral screening agents are chosen from pigments or nanopigments (mean size of the primary particles: generally between 5 nm and 100 nm and preferably between 10 nm and 50 nm) of coated or uncoated metal oxides such as, for example, nanopigments of titanium oxide (amorphous or crystallized in rutile and/or anatase form), of iron oxide, of zinc oxide, of zirconium oxide or of cerium oxide, which are all UV-photoprotectors that are well known per se. Standard coating agents are, moreover, alumina and/or aluminum stearate. Such coated or uncoated metal oxide nanopigments are described in particular in EP-518,772 and EP-518,773.

The additional UV-screening agents in accordance with the invention are generally present in the compositions according to the invention in proportions ranging from 0.01% to 20% by weight relative to the total weight of the composition, and preferably ranging from 0.1% to 10% by weight relative to the total weight of the composition.

The cosmetic compositions according to the invention may also contain agents for artificially tanning and/or browning the skin (self-tanning agents) such as dihydroxyacetone (DHA).

The compositions in accordance with the present invention may also comprise standard cosmetic adjuvants chosen especially from fatty substances, organic solvents, ionic or nonionic thickeners, softeners, humectants, antioxidants, moisturizers, desquamating agents, free-radical scavengers, antipollution agents, antibacterial agents, anti-inflammatory agents, depigmenting agents, propigmenting agents, opacifiers, stabilizers, emollients, silicones, antifoams, insect repellents, fragrances, preservatives, anionic, cationic, nonionic, zwitterionic or amphoteric surfactants, substance P antagonists, substance CGRP antagonists, fillers, pigments, polymers, propellants, acidifying or basifying agents, or any other ingredient usually used in cosmetics and/or dermatology.

The fatty substances may consist of an oil or a wax or mixtures thereof. The term “oil” means a compound that is liquid at room temperature. The term “wax” means a compound that is solid or substantially solid at room temperature, and whose melting point is generally above 35° C.

Oils that may be mentioned include mineral oils (paraffin); plant oils (sweet almond oil, macadamia oil, blackcurrant seed oil or jojoba oil); synthetic oils, for instance perhydrosqualene, fatty alcohols, fatty acids or fatty acid esters (for instance the C₁₂-C₁₅ alcohol benzoate sold under the trademark “Finsolv TN” by Witco, octyl palmitate, isopropyl lanolate, triglycerides, including capric/caprylic acid triglycerides), oxyethylenated or oxypropylenated fatty esters and ethers; silicone oils (cyclomethicone and polydimethylsiloxanes, or PDMS) or fluoro oils, and polyalkylenes.

Waxy compounds that may be mentioned include paraffin, carnauba wax, beeswax and hydrogenated castor oil.

Among the organic solvents that may be mentioned are lower alcohols and polyols. These lower polyols may be chosen from glycols and glycol ethers, for instance ethylene glycol, propylene glycol, butylene glycol, dipropylene glycol or diethylene glycol.

The thickeners may be chosen especially from crosslinked acrylic polymers, for instance Carbomers, acrylate/C₁₀-C₃₀ alkylacrylate crosslinked polymers of the type such as Pemulen or polyacrylate-3 sold under the name Viscophobe DB 1000 by Amerchol; poly-acrylamides such as the emulsion of polyacrylamide, C₁₃-C₁₄ isoparaffin and laureth-7 sold under the name Sepigel 305 by SEPPIC, AMPS homopolymers or copolymers such as Hostacerin AMPS sold by Clariant, modified or unmodified guar gums and celluloses, such as hydroxy-propyl guar gum, methylhydroxyethylcellulose and hydroxypropylmethyl cellulose, xanthan gum, and nanometric silicas of the Aerosil type.

Needless to say, a person skilled in the art will take care to select the optional additional compound(s) mentioned above and/or the amounts thereof such that the advantageous properties intrinsically associated with the compositions in accordance with the invention are not, or are not substantially, adversely affected by the envisaged addition(s).

The compositions according to the invention may be prepared according to techniques that are well known to those skilled in the art, in particular those intended for the preparation of emulsions of oil-in-water or water-in-oil type.

This composition may be in particular in the form of a simple or complex emulsion (O/W, W/O, O/W/O or W/O/W emulsion) such as a cream or a milk, in the form of a gel or a cream-gel, or in the form of a lotion, an oil, a powder or a solid tube, and may optionally be packaged as an aerosol and may be in the form of a mousse or a spray.

The compositions according to the invention are preferably in the form of an oil-in-water or water-in-oil emulsion.

When it is an emulsion, the aqueous phase of this emulsion may comprise a nonionic vesicular dispersion prepared according to known processes (Bangham, Standish and Watkins, J. Mol. Biol. 13, 238 (1965), FR-2-315,991 and FR-2-416,008).

When the cosmetic composition according to the invention is used to care for the human epidermis, it may be in the form of a suspension or a dispersion in solvents or fatty substances, in the form of a nonionic vesicular dispersion or in the form of an emulsion, preferably of oil-in-water type, such as a cream or a milk, or in the form of an ointment, a gel, a cream-gel, an antisun oil, a solid tube, a powder, an aerosol mousse or a spray.

When the cosmetic composition according to the invention is used for haircare, it may be in the form of a shampoo, a lotion, a gel, an emulsion or a nonionic vesicular dispersion and may constitute, for example, a rinse-out composition, to be applied before or after shampooing, before or after dyeing or bleaching, or before, during or after permanent-waving or relaxing the hair, a styling or treating lotion or gel, a blow-drying or hairsetting lotion or gel, or a composition for permanent-waving, relaxing, dyeing or bleaching the hair.

When the composition is used as a makeup product for the nails, the lips, the eyelashes, the eyebrows or the skin, such as an epidermal treatment cream, a foundation, a tube of lipstick, an eyeshadow, a makeup rouge, a mascara or an eyeliner, it may be in solid or pasty, anhydrous or aqueous form, such as oil-in-water or water-in-oil emulsions, nonionic vesicular dispersions or suspensions.

As a guide, for the antisun formulations in accordance with the invention which contain a support of oil-in-water emulsion type, the aqueous phase (especially comprising the hydrophilic screening agents) generally represents from 50% to 95% by weight and preferably from 70% to 90% by weight relative to the total weight of the formulation, the oily phase (especially comprising the lipophilic screening agents) generally represents from 5% to 50% by weight and preferably from 10% to 30% by weight relative to the total weight of the formulation, and the (co)-emulsifier(s) generally represent(s) from 0.5% to 20% by weight and preferably from 2% to 10% by weight relative to the total weight of the formulation.

A subject of the present invention is also the use of the said n-butylphthalimide/isopropylphthalimide eutectic mixture in a cosmetic or dermatological composition containing a 1,3,5-triazine derivative as solvent for the said triazine derivative in the said composition.

The present invention also relates to the use of the said n-butylphthalimide/isopropylphthalimide eutectic mixture in a cosmetic or dermatological composition containing a 1,3,5-triazine derivative, in order to improve the sun protection factor, the cosmetic properties and/or the stability of this composition.

In order to further illustrate the present invention and the advantages thereof, the following specific examples are given, it being understood that same are intended only as illustrative and in nowise limitative. In said examples to follow, all parts and percentages are given by weight, unless otherwise indicated.

### EXAMPLES

Comparative Study of the Solubility of a Triazine Derivative in Different Solvents

The solubility of a triazine derivative (i.e., 2,4,6-tris(diisobutyl 4′-aminobenzalmalonate)-s-triazine) in different solvents was determined by means of the following test:

20 g of solvent are added, at room temperature, to 5 g of screening agent,

the mixture thus obtained is brought to 83° C. using a water bath,

the presence or absence of crystals is observed after 24 hours and cooling to room temperature.

The results obtained are collated in the table below (the chemical names are given in the CTFA nomenclature, 5th edition, 1993).

| | SOLUBILITY |
|---|---|
| | OF THE TRIAZINE |
| SOLVENT | SCREENING AGENT |
| n-butylphthalimide/isopropylphthalimide | 40% |
| eutectic mixture | |
| Pelemol BIP from Phoenix Chemicals | |
| (invention) | |
| C₈-C₁₂ fatty acid triglycerides | <10% |
| “Miglyol 812” from Huls | |
| PPG-3 myristyl ether | <10% |
| “Witconol APM” from Witco | |
| C₁₂-C₁₅ alkyl benzoate | <10% |
| “Finsolv TN” from Finetex | |

These results clearly demonstrate the greater solubilizing power of the eutectic mixture of n-butyl-phthalimide/isopropylphthalimide in accordance with the invention and the noteworthy beneficial effect afforded by the presence of oil as regards the solubility in the final compositions.

Test of In Vitro Efficacy:

The method for evaluating the protection factor used is the in vitro method described by B. L. Diffey et al. in J. Soc. Cosmet. Chem. 40-127-133 (1989), which consists in determining the monochromatic protection factors every 5 nm in a wavelength range from 290 to 400 nm, and in calculating therefrom the sun protection factor according to a given mathematical equation.

Results:

Two screening agents of triazine type were formulated in a support of O/W type, at 5% by weight, in the solvent according to the invention and in a comparative solvent (Finsolv TN), and three SPF values, the mean and the standard deviation of which are collated in the table below, were determined for each formulation:

Composition

| | |
|---|---|
| 80/20 mixture of cetylstearyl alcohol and | 7 |
| of oxyethylenated (33 EO) cetylstearyl alcohol | |
| (Sinnowax AO - Henkel) | |
| Mixture of glyceryl monostearate and distearate | 2 |
| (Cerasynt SD-V ISP) | |
| Cetyl alcohol | 1.5 |
| Polydimethylsiloxane | 1.5 |
| (Dow Corning 200 Fluid - Dow Corning) | |
| Solvent | 15 |
| Screening Agent of Triazine Type | 5 |
| glycerol | 20 |
| triethanolamine | qs pH 7 |
| preservatives | qs |
| demineralized water | qs 100 g |

| | | Mean SPF |
|---|---|---|
| | | (Standard |
| Screening Agents (5%) | Solvents (15%) | Deviation) |
| 2,4,6-tris(di-iso-butyl 4′- | n-butylphthalimide/-iso- | 8.0 |
| amino-benzal-malonate)- | propyl-phthalimide | (0.7) |
| s-triazine | Pelemol BIP from | |
| | Phoenix Chemicals | |
| | (invention) | |
| | C₁₂-C₁₅ alkyl benzoate | 4.1 |
| | “Finsolv TN” from | (0.1) |
| | Finetex | |
| 2,4-bis{[4-ethyl-2-hexyloxy)]- | n-butylphthalimide/iso- | 11.4 |
| 2-hydroxy]-phenyl}- | propyl-phthalimide | (2) |
| 6-(4-meth-oxy-phenyl)- | Pelemol BIP from | |
| 1,3,5-triazine | Phoenix Chemicals | |
| “Anisotriazine” sold | (invention) | |
| under the trademark | C₁₂-C₁₅ alkyl benzoate | 8.5 |
| “Tinosorb S” by Ciba | “Finsolv TN” from | (1.4) |
| Specialty Chemicals | Finetex | |

For the same photoprotective system, the emulsion according to the invention containing the n-butylphthalimide/isopropylphthalimide eutectic mixture as solvent has a significantly higher SPF value.

Each patent, patent application, publication and literature article/report cited or indicated herein is hereby expressly incorporated by reference.

While the invention has been described in terms of various specific and preferred embodiments, the skilled artisan will appreciate that various modifications, substitutions, omissions, and changes may be made without departing from the spirit thereof. Accordingly, it is intended that the scope of the present invention be limited solely by the scope of the following claims, including equivalents thereof.

What is claimed is:

1. A topically applicable cosmetic/dermatological composition suited for the UV-protection of the skin, hair, lips and/or integuments, comprising (i) an effective UV-photoprotecting amount of at least one 1,3,5-triazine sunscreen, and (ii) as a solubilizing agent therefor, a thus effective amount of at least one mixed phthalimide eutectic.

2. The cosmetic/dermatological composition as defined by claim 1, said at least one mixed phthalimide eutectic comprising (a) n-butylphthalimide having the structural formula (1): 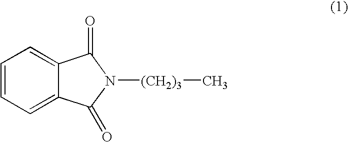
and (b) isopropylphthalimide having the structural formula (2):

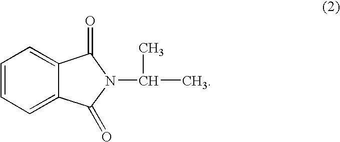

3. The cosmetic/dermatological composition as defined by claim 2, said at least one mixed phthalimide eutectic comprising from 60% to 75% by weight of said n-butylphthalimide.

4. The cosmetic/dermatological composition as defined by claim 3, said at least one mixed phthalimide eutectic being selected from the group consisting of:
n-butylphthalimide/isopropylphthalimide (60/40% by weight),
n-butylphthalimide/isopropylphthalimide (62/38% by weight),
n-butylphthalimide/isopropylphthalimide (65/35% by weight),
n-butylphthalimide/isopropylphthalimide (70/30% by weight), and
n-butylphthalimide/isopropylphthalimide (75/25% by weight).

5. The cosmetic/dermatological composition as defined by claim 2, said at least one mixed phthalimide eutectic comprising from 0.1% to 50% by weight thereof.

6. The cosmetic/dermatological composition as defined by claim 2, said at least one mixed phthalimide eutectic comprising from 1% to 30% by weight thereof.

7. The cosmetic/dermatological composition as defined by claim 2, said at least one 1,3,5-triazine sunscreen having the structural formula (I): 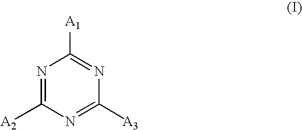
in which the radicals A₁, A₂ and A₃, which may be identical or different, are each a radical of formulae (II) to (IX) below: 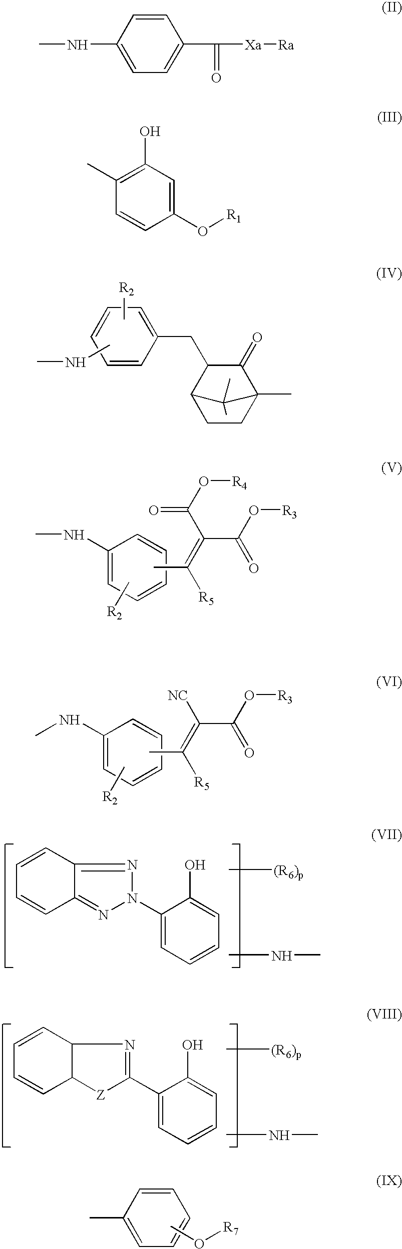
in which each of the groups Xₐ, which may be identical or different, is oxygen or —NH—; each of the groups Rₐ, which may be identical or different, is hydrogen; an alkali metal; an ammonium radical optionally substituted with one or more linear or branched C₁-C₁₈ alkyl or linear or branched C₁-C₁₈ hydroxyalkyl radicals; a linear or branched C₁-C₁₈ radical; a C₅-C₁₂ cycloalkyl radical optionally substituted with one or more C₁-C₄ alkyl radicals; a polyoxyethylenated radical comprising from 1 to 6 ethylene oxide units, the terminal OH group of which is methylated; a radical of formula (X), (XI) or (XII) below: 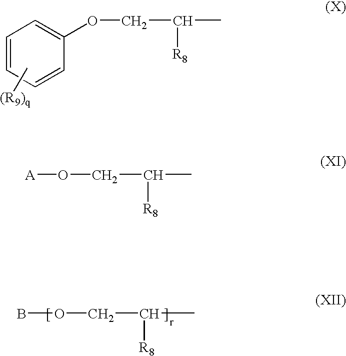
in which R₈ is hydrogen or a methyl radical; R₉ is a C₁-C₉ alkyl radical; q is an integer equal to O; 1; 2; 3; r is an integer equal to 1; 2; 3; 4; 5; 6; 7; 8; 9; 10; A is a C₄-C₈ alkyl radical or a C₅-C₈ cycloalkyl radical; B is a linear or branched C₁-C₈ alkyl radical; a C₅-C₈ cycloalkyl radical; an aryl radical optionally substituted with one or more C₁-C₄ alkyl radicals; R₁ is a C₃-C₁₈ alkyl radical; a C₂-C₁₈ alkenyl radical; a radical of formula —CH₂—CH(OH)—CH₂—OT₁ in which T₁ is a hydrogen atom or a C₁-C₈ alkyl radical; a radical of formula (XIII) below: 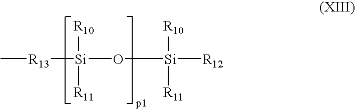
in which R₁₃ is a covalent bond; a linear or branched C₁-C₄ alkyl radical or a radical of formula —Cₘ₁H₂ₘ₁—O— in which m₁ is an integer equal to 1; 2; 3; 4; p₁ is an integer equal to 0; 1; 2; 3; 4; 5; the radicals R₁₀, R₁₁ and R₁₂, which may be identical or different, are each a C₁-C₁₈ alkyl radical; a C₁-C₁₈ alkoxy radical or a radical of formula: 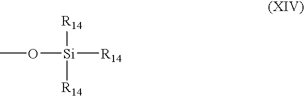
in which R₁₄ is a C₁-C₁₀ alkyl radical; R₂ is a hydrogen atom, a linear or branched C₁-C₄ alkyl radical or a C₁-C₄ alkoxy radical; R₃ and R₄, which may be identical or different, are each a linear or branched C₁-C₂₀ alkyl radical; R₅ is a hydrogen atom or a phenyl radical optionally substituted with a halogen or with a C₁-C₄ alkyl radical or with a C₁-C₄ alkoxy radical; R₆ is a linear or branched C₁-C₈ alkyl radical or a C₁-C₃ alkoxy radical, with the proviso that, in the latter instance, two adjacent radicals R₆ on the same aromatic nucleus can together form an alkylidenedioxy group in which the alkylidene group contains 1 or 2 carbon atoms, OH, NHCOCH₃ or NH₂; R₇ is a hydrogen atom, a C₁-C₁₀ alkyl radical, a radical of formula: —(CH₂CHR₅—O)ₙ₁R₈ in which n1 is a number ranging from 1 to 16, or a radical of structure —CH₂—CH—(OH)—CH₂OT, in which R₈ and T₁ are as defined above; Z is oxygen, sulfur, —NH— or —NR₃— in which R₃ is a linear or branched C₁-C₂₀ alkyl radical; p is 0, 1, 2 or 3; A₁ can also be a halogen, a radical —N(R₃)₂, the two radicals R₃ together optionally forming a ring of 4 or 5 carbon atoms, or a group —OR₃ in which R₃ is as defined above.

8. The cosmetic/dermatological composition as defined by claim 7, said at least one 1,3,5-triazine sunscreen having the structural formula (I) in which A₁, A₂ and A₃ are of formula (II) and have all of the following characteristics:
one of the radicals Xₐ—Rₐ is a radical-NH—Rₐ in which Rₐ is a C₅-C₁₂ cycloalkyl radical optionally substituted with one or more C₁-C₄ alkyl radicals; a radical of formula (X), (XI) or (XII) above in which:
B is a C₁-C₄ alkyl radical;
R₉ is a methyl radical;
the other two radicals Xₐ—Rₐ are each a radical —O—Rₐ in which the radicals Rₐ, which may be identical or different, are each hydrogen; an alkali metal; an ammonium radical optionally substituted with one or more alkyl or hydroxyalkyl radicals; a linear or branched C₁-C₁₈ alkyl radical; a C₅-C₁₂ cycloalkyl radical optionally substituted with one or more C₁-C₄ alkyl radicals; a radical of formula (X), (XI) or (XII) above in which:
B is a C₁-C₄ alkyl radical; and
R₉ is a methyl radical.

9. The cosmetic/dermatological composition as defined by claim 7, said at least one 1,3,5-triazine sunscreen having the structural formula (I) in which A₁, A₂ and A₃ are of formula (II) and have all of the following characteristics:
one or two radicals Xₐ—Rₐ is a radical —NH—Rₐ, in which Rₐ is a linear or branched C₁-C₁₈ alkyl radical; a C₅-C₁₂ cycloalkyl radical optionally substituted with one or more C₁-C₄ alkyl radicals; a radical of formula (X), (XI) or (XII) above in which:
B is a C₁-C₄ alkyl radical;
R₉ is a methyl radical;
the other or the other two radical(s) Xₐ—Rₐ being a radical —O—Rₐ in which the radicals Rₐ, which may be identical or different, are each hydrogen; an alkali metal; an ammonium radical optionally substituted with one or more alkyl or hydroxyalkyl radicals; a linear or branched C₁-C₁₈ alkyl radical; a C₅-C₁₂ cycloalkyl radical optionally substituted with one or more C₁-C₄ alkyl radicals; a radical of formula (X), (XI) or (XII) above in which:
B is a C₁-C₄ alkyl radical; and
R₉ is a methyl radical.

10. The cosmetic/dermatological composition as defined by claim 9, said at least one 1,3,5-triazine sunscreen comprising 2-[(p-(tert-butylamido)-anilino]-4,6-bis[(p-(2′-ethylhexyl-1′-oxycarbonyl)-anilino]-1,3,5-triazine, having the following structural formula: 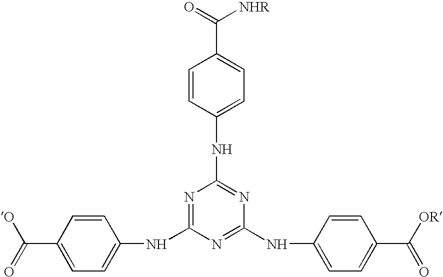
in which R′ is a 2-ethylhexyl radical and R is a tert-butyl radical.

11. The cosmetic/dermatological composition as defined by claim 7, said at least one 1,3,5-triazine sunscreen having the structural formula (I) in which A₁, A₂ and A₃ are of formula (II) and have all of the following characteristics:
the radicals Xₐ are identical and each is oxygen;
the radicals Rₐ which may be identical or different, are each a C₆-C₁₂ alkyl radical or a polyoxyethylenated radical comprising from 1 to 6 ethylene oxide units, the terminal OH group of which is methylated.

12. The cosmetic/dermatological composition as defined by claim 11, said at least one 1,3,5-triazine sunscreen comprising 2,4,6-tris[p(2′-ethylhexyl-1′-oxycarbonyl)anilino]-1,3,5-triazine, having the following structural formula: 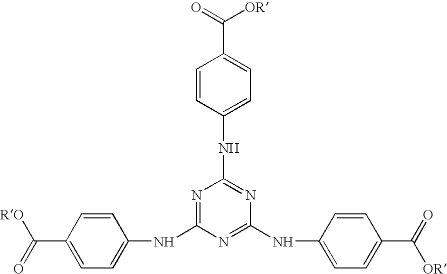
in which R′ is a 2-ethylhexyl radical.

13. The cosmetic/dermatological composition as defined by claim 7, said at least one 1,3,5-triazine sunscreen having the structural formula (I) in which A₁ and A₂ are of formula (III) and A₃ is of formula (IX) and have all of the following characteristics:
the radicals R₁, which may be identical or different, are each a C₃-C₁₈ alkyl radical; a C₂-C₁₈ alkenyl radical or a radical of formula —CH₂—CH(OH)—CH₂—OT₁ in which T₁ is a hydrogen atom or a C₁-C₈ alkyl radical; and R₇ is a hydrogen atom or a C₁-C₁₀ alkyl radical.

14. The cosmetic/dermatological composition as defined by claim 13, said at least one 1,3,5-triazine sunscreen comprising 2,4-bis{[4-2-ethylhexyloxy)]-2-hydroxy]phenyl}-6-(4-methoxyphenyl)-1,3,5-triazine, having the following structural formula: 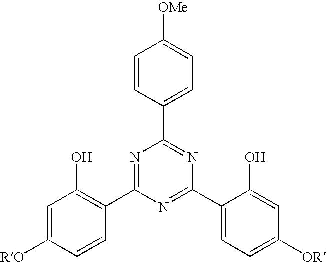
in which R′ is a 2-ethylhexyl radical.

15. The cosmetic/dermatological composition as defined by claim 7, said at least one 1,3,5-triazine sunscreen having the structural formula (I) in which A₁, A₂ and A₃ are of formulae (VII) to (XI).

16. The cosmetic/dermatological composition as defined by claim 15, said at least one 1,3,5-triazine sunscreen being selected from the group consisting of:
2,4,6-tris(diisobutyl 4′-aminobenzalmalonate)-s-triazine,
2,4,6-tris(bis(2-ethylhexyl) 4′-aminobenzal-malonate)-s-triazine,
2,4,6-tris(bis(2-ethylhexyl) 4′-aminobenzal-malonate)-6-chloro-s-triazine,
2,4,6-tris(bis(2-ethylhexyl) 4′-aminobenzal-malonate)-6-(2-ethylhexyl 4′-aminobenzoate)-s-triazine,
2,4,6-tris(diisobutyl 4′-aminobenzalmalonate)-6-butoxy-s-triazine,
2,4,6-tris(diisobutyl 4′-aminobenzalmalonate)-6-(2-ethylhexylamino)-s-triazine,
2,4-bis(4′-aminobenzylidenecamphor)-6-(2-ethyl-hexylamino)-s-triazine,
2,4-bis(4′-aminobenzylidenecamphor)-6-(diisobutyl 4′-aminobenzalmalonate)-s-triazine,
2,4,6-tris(diethyl 4′-aminobenzalmalonate)-s-triazine,
2,4,6-tris(diisopropyl 4′-aminobenzalmalonate)-s-triazine,
2,4,6-tris(dimethyl 4′-aminobenzalmalonate)-s-triazine,
2,4,6-tris(ethyl a-cyano-4-aminocinnamate)-s-triazine,
2,4,6-tris[(3′-benzotriazol-2-yl-2′-hydroxy-5′-methyl)phenylamino]-s-triazine, and
2,4,6-tris[(3′-benzotriazol-2-yl-2′-hydroxy-5′-tert-octyl)phenylamino]-s-triazine.

17. The cosmetic/dermatological composition as defined by claim 16, said at least one 1,3,5-triazine sunscreen comprising 2,4,6-tris(diisobutyl 4′-aminobenzalmalonate)-s-triazine.

18. The cosmetic/dermatological composition as defined by claim 2, said at least one 1,3,5-triazine sunscreen comprising from 0.05% to 15% by weight thereof.

19. The cosmetic/dermatological composition as defined by claim 2, said at least one 1,3,5-triazine sunscreen comprising from 0.1% to 10% by weight thereof.

20. The cosmetic/dermatological composition as defined by claim 2, further comprising at least one additional organic UV-screening agent and/or at least one additional mineral UV-screening agent, which is water-soluble, liposoluble or insoluble in a conventional cosmetic solvent.

21. The cosmetic/dermatological composition as defined by claim 20, comprising at least one additional organic UV-screening agent selected from the group consisting of anthranilates; cinnamic derivatives; dibenzoylmethane derivatives; salicylic derivatives; camphor derivatives; benzophenone derivatives; β,β-diphenyl-acrylate derivatives; benzotriazole derivatives; benzalmalonate derivatives; benzimidazole derivatives; imidazolines; bis-benzazolyl derivatives; p-amino-benzoic acid (PABA) derivatives; methylenebis(hydroxy-phenylbenzotriazole) derivatives; benzoxazole derivatives; UV-screening polymers and UV-screening silicones; dimers derived from α-alkyl-styrene; 4,4-diarylbutadienes, and mixtures thereof.

22. The cosmetic/dermatological composition as defined by claim 21, further comprising at least one additional organic UV-screening agent selected from the group consisting of:
ethylhexyl salicylate,
ethylhexyl methoxycinnamate,
octocrylene,
phenylbenzimidazolesulfonic acid,
benzophenone-3,
benzophenone-4,
benzophenone-5,
n-hexyl 2-(4-diethylamino-2-hydroxybenzoyl)-benzoate,
4-methylbenzylidenecamphor,
terephthalylidenedicamphorsulfonic acid,
disodium phenyldibenzimidazoletetrasulfonate,
methylenebis(benzotriazolyl)tetramethylbutylphenol,
Drometrizole trisiloxane,
Polysilicone-15
1,1-dicarboxy(2,2′-dimethylpropyl)-4,4-diphenyl-butadiene,
2,4-bis-[5-1 (diméthylpropyl)benzoxazol-2-yl-(4-phenyl)-imino]-6-(2-ethylhexyl)-imino-1,3,5-triazine
and mixtures thereof.

23. The cosmetic/dermatological composition as defined by claim 20, comprising at least one additional mineral UV-screening agent selected from among coated or uncoated metal oxide pigments or nanopigments.

24. The cosmetic/dermatological composition as defined by claim 23, said at least one additional mineral UV-screening agent comprising nanopigments of titanium oxide, amorphous or crystallized in rutile and/or anatase form, of iron oxide, of zinc oxide, of zirconium oxide or of cerium oxide.

25. The cosmetic/dermatological composition as defined by claim 20, said at least one additional UV-screening agent comprising from 0.1% to 20% by weight thereof.

26. The cosmetic/dermatological composition as defined by claim 20, said at least one additional UV-screening agent comprising from 0.2% to 15% by weight thereof.

27. The cosmetic/dermatological composition as defined by claim 2, further comprising at least one cosmetic adjuvant selected from the group consisting of fatty substances, organic solvents, emulsifiers, ionic or nonionic thickeners, softeners, antioxidants, free-radical scavengers, opacifiers, stabilizers, emollients, silicones, a-hydroxy acids, antifoams, moisturizers, vitamins, insect repellents, fragrances, preservatives, surfactants, anti-inflammatory agents, substance P antagonists, fillers, polymers, propellants, acidifying or basifying agents and dyes/colorants.

28. The cosmetic/dermatological composition as defined by claim 2, formulated as a suspension or dispersion, a nonionic vesicular dispersion, an emulsion, an ointment, a gel, a cream-gel, a solid tube, a powder, wipes, an aerosol mousse or a spray.

29. The cosmetic/dermatological composition as defined by claim 2, formulated as a makeup composition for the eyelashes, the eyebrows, the nails or the skin and comprising a solid or a paste, whether anhydrous or aqueous.

30. The cosmetic/dermatological composition as defined by claim 2, formulated for protecting the hair against ultraviolet radiation and comprising a shampoo, a lotion, a gel, an emulsion or a nonionic vesicular dispersion.

31. A regime or regimen for UV-photoprotecting the skin, hair, lips and/or integuments against the damaging effects of UV radiation, comprising topically applying thereon a thus effective amount of the cosmetic/dermatological composition as defined by claim 1.

32. A regime or regimen for UV-photoprotecting the skin, hair, lips and/or integuments against the damaging effects of UV radiation, comprising topically applying thereon a thus effective amount of the cosmetic/dermatological composition as defined by claim 2.

33. A method for improving the sun protection factor, the cosmetic properties and/or the stability of a cosmetic/dermatological composition suited for UV-photoprotecting the skin, hair, lips and/or integuments against the damaging effects of UV radiation and comprising at least one 1,3,5-triazine sunscreen, comprising formulating therewith at least one n-butylphthalimide/isopropylphthalimide eutectic mixture.

34. A topically applicable cosmetic/dermatological composition suited for UV-photoprotecting the skin, hair, lips and/or integuments against the damaging effects of UV radiation, which comprises (i) an effective UV-photoprotecting amount of at least one 1,3,5-triazine sunscreen, and (ii) an amount of at least one eutectic mixture sufficient to dissolve all of said at least one 1,3,5-triazine sunscreen, said at least one eutectic mixture comprising (a) n-butylphthalimide having the structural formula (1): 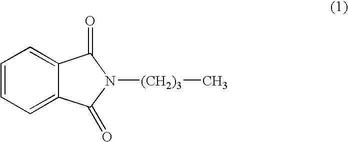
and (b) isopropylphthalimide having the structural formula (2): 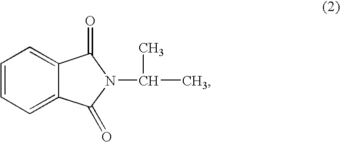
formulated into a topically applicable, cosmetically/dermatologically acceptable medium therefor.
